SEQUENCE LISTING (92 sequences)

SEQ ID NO: 1 - DNA, 23 bp, synthetic construct
  source 1..23: mol_type = other DNA
tgtttcctcatctcacggtaagg

SEQ ID NO: 2 - DNA, 23 bp, synthetic construct
  source 1..23: mol_type = other DNA
aatgtacagtagttctccttcgg

SEQ ID NO: 3 - DNA, 23 bp, synthetic construct
  source 1..23: mol_type = other DNA
tcacagctgctgccttaagcagg

SEQ ID NO: 4 - DNA, 23 bp, synthetic construct
  source 1..23: mol_type = other DNA
cccagcactcaaaggaccgaagg

SEQ ID NO: 5 - RNA, 20 nt, synthetic construct
  source 1..20: mol_type = other RNA
tgtttcctcatctcacggta

SEQ ID NO: 6 - RNA, 20 nt, synthetic construct
  source 1..20: mol_type = other RNA
aatgtacagtagttctcctt

SEQ ID NO: 7 - RNA, 20 nt, synthetic construct
  source 1..20: mol_type = other RNA
tcacagctgctgccttaagc

SEQ ID NO: 8 - RNA, 20 nt, synthetic construct
  source 1..20: mol_type = other RNA
cccagcactcaaaggaccga

SEQ ID NO: 9 - DNA, 24 bp, synthetic construct
  source 1..24: mol_type = other DNA
ttttctcctagacatcatgccaca

SEQ ID NO: 10 - DNA, 25 bp, synthetic construct
  source 1..25: mol_type = other DNA
ccagtggaacaagtactgaaagcta

SEQ ID NO: 11 - DNA, 23 bp, synthetic construct
  source 1..23: mol_type = other DNA
caacgtcgtatctccgataacgg

SEQ ID NO: 12 - DNA, 23 bp, synthetic construct
  source 1..23: mol_type = other DNA
ctaactaagccataccatactgg

SEQ ID NO: 13 - DNA, 23 bp, synthetic construct
  source 1..23: mol_type = other DNA
atcatcaccacgatgttgttggg

SEQ ID NO: 14 - DNA, 23 bp, synthetic construct
  source 1..23: mol_type = other DNA
ccagcagggcatcagaatacagg

SEQ ID NO: 15 - RNA, 20 nt, synthetic construct
  source 1..20: mol_type = other RNA
caacgtcgtatctccgataa

SEQ ID NO: 16 - RNA, 20 nt, synthetic construct
  source 1..20: mol_type = other RNA
ctaactaagccataccatac

SEQ ID NO: 17 - RNA, 20 nt, synthetic construct
  source 1..20: mol_type = other RNA
atcatcaccacgatgttgtt

SEQ ID NO: 18 - RNA, 20 nt, synthetic construct
  source 1..20: mol_type = other RNA
ccagcagggcatcagaatac

SEQ ID NO: 19 - DNA, 24 bp, synthetic construct
  source 1..24: mol_type = other DNA
aacacactgtagctgatacgtctt

SEQ ID NO: 20 - DNA, 25 bp, synthetic construct
  source 1..25: mol_type = other DNA
ctaaaccacaggacagtttactcac

SEQ ID NO: 21 - DNA, 23 bp, synthetic construct
  source 1..23: mol_type = other DNA
gaagttgaaacctggtattttgg

SEQ ID NO: 22 - DNA, 23 bp, synthetic construct
  source 1..23: mol_type = other DNA
ccactacagcaactttccaaagg

SEQ ID NO: 23 - RNA, 20 nt, synthetic construct
  source 1..20: mol_type = other RNA
gaagttgaaacctggtattt

SEQ ID NO: 24 - RNA, 20 nt, synthetic construct
  source 1..20: mol_type = other RNA
ccactacagcaactttccaa

SEQ ID NO: 25 - DNA, 25 bp, synthetic construct
  source 1..25: mol_type = other DNA
gcttagaggtccaatttcttggtct

SEQ ID NO: 26 - DNA, 25 bp, synthetic construct
  source 1..25: mol_type = other DNA
gttgttcccttctagtgttttctcg

SEQ ID NO: 27 - DNA, 23 bp, synthetic construct
  source 1..23: mol_type = other DNA
gggcagccaaaagcatctgatgg

SEQ ID NO: 28 - DNA, 23 bp, synthetic construct
  source 1..23: mol_type = other DNA
tatacagcatgatgcaaccttgg

SEQ ID NO: 29 - RNA, 20 nt, synthetic construct
  source 1..20: mol_type = other RNA
gggcagccaaaagcatctga

SEQ ID NO: 30 - RNA, 20 nt, synthetic construct
  source 1..20: mol_type = other RNA
tatacagcatgatgcaacct

SEQ ID NO: 31 - DNA, 25 bp, synthetic construct
  source 1..25: mol_type = other DNA
accttgaggctactatgctttactc

SEQ ID NO: 32 - DNA, 24 bp, synthetic construct
  source 1..24: mol_type = other DNA
tcaatccaaatggatgcagaacac

SEQ ID NO: 33-44 - skipped (12 entries)

SEQ ID NO: 45 - DNA, 23 bp, synthetic construct
  source 1..23: mol_type = other DNA
aaaagagtacctgatgacacagg

SEQ ID NO: 46 - DNA, 23 bp, synthetic construct
  source 1..23: mol_type = other DNA
cagctgatggatgcacagattgg

SEQ ID NO: 47 - RNA, 20 nt, synthetic construct
  source 1..20: mol_type = other RNA
aaaagagtacctgatgacac

SEQ ID NO: 48 - RNA, 20 nt, synthetic construct
  source 1..20: mol_type = other RNA
cagctgatggatgcacagat

SEQ ID NO: 49 - DNA, 21 bp, synthetic construct
  source 1..21: mol_type = other DNA
acatggcaacacatgcaaata

SEQ ID NO: 50 - DNA, 27 bp, synthetic construct
  source 1..27: mol_type = other DNA
ctgactcaatacatatgctctgtaaac

SEQ ID NO: 51-86 - skipped (36 entries)

SEQ ID NO: 87 - DNA, 23 bp, synthetic construct
  source 1..23: mol_type = other DNA
attggcacagctcatcagtgtgg

SEQ ID NO: 88 - DNA, 23 bp, synthetic construct
  source 1..23: mol_type = other DNA
ggttttgggtactgacacagagg

SEQ ID NO: 89 - RNA, 20 nt, synthetic construct
  source 1..20: mol_type = other RNA
attggcacagctcatcagtg

SEQ ID NO: 90 - RNA, 20 nt, synthetic construct
  source 1..20: mol_type = other RNA
ggttttgggtactgacacag

SEQ ID NO: 91 - DNA, 17 bp, synthetic construct
  source 1..17: mol_type = other DNA
cagggcagctgctgaaa

SEQ ID NO: 92 - DNA, 20 bp, synthetic construct
  source 1..20: mol_type = other DNA
aagacaatcggccaactctg

(19) United States
(12) Patent Application Publication
(10) Pub. No.: US 2026/0218123 A1
(43) Pub. Date: Jul. 30, 2026

(54) ETHICAL TISSUES FOR TRANSPLANTATION

(71) Applicant: Kind Biotechnology Inc., Derry, NH (US)

(72) Inventors: Justin REBO, Derry, NH (US); Timothy WEBB, Manchester, NH (US)

(21) Appl. No.: 19/147,271

(22) PCT Filed: Jan. 9, 2024
(86) PCT No.: PCT/US2024/010951

§ 371 Date: Jul. 10, 2025

Related U.S. Application Data
(60) Provisional application No. 63/479,225, filed on Jan. 10, 2023.
(60) Provisional application No. 63/480,234, filed on Jan. 17, 2023.
(60) Provisional application No. 63/439,633, filed on Jan. 18, 2023.

(51) Int. Cl. C12N 5/073; A01K 67/0275

(52) U.S. Cl. CPC C12N 5/0604; A01K 67/0275; A01K 2217/15; A01K 2267/025; C12N 2510/00; C12N 2511/00; C12N 2513/00

(57) ABSTRACT

The present disclosure relates to organisms genetically engineered to have reduced higher brain structures, tissues obtained from such organisms, and methods of making such organisms for the purpose of supplying cruelty free tissues and organs from organisms with severely reduced ability to suffer or completely lack the ability to experience suffering.

### 1. CROSS-REFERENCE TO RELATED APPLICATIONS

This application claims the benefit of and priority to U.S. Provisional Application Nos. 63/479,225, filed Jan. 10, 2023, 63/480,234, filed Jan. 17, 2023, and 63/439,633, filed Jan. 18, 2023, which are hereby incorporated by reference in their entirety.

### 2. SEQUENCE LISTING

The instant application contains a Sequence Listing which has been submitted electronically via Patent Center and is hereby incorporated by reference in its entirety. Said XML copy, created on Month XX, 2024, is named 54661WO_sequencelisting.xml, and is X,XXX,XXX bytes in size.

### 3. BACKGROUND OF THE INVENTION

There is a need for an alternative to current practices used in raising, slaughtering, and processing animals for harvest of meat and other tissues, including complex organs, one that does not cause pain and suffering to the involved animals.

### 4. SUMMARY OF THE INVENTION

An approach that guarantees a lack of sentience in animals intended for harvest of tissues would be an ideal approach to reduction in pain and suffering of the involved animals. The present strategy offers an approach to production of tissues, including organs for transplantation, through the creation of organisms that cannot develop higher brain function. In some embodiments, the engineered organisms possess enough nervous tissue to maintain basic organ function, but lack any ability for conscious awareness. In other embodiments, lower brain function is also reduced or eliminated, and the tissues and organs are kept alive through artificial means.

Engineering the organism's body without the brain, or parts of the brain, allows the harvesting of tissues while solving the critical issues of pain and suffering that are inherent in raising livestock. Non-meat animal products, such as leather, casings, and even organs for transplantation, can likewise be produced without pain and suffering. Moreover, by eliminating the need for traditional animal agriculture and associated land use, and by keeping the animal without higher brain function in a controlled environment, methane and other emissions can easily be captured; engineering animals without higher brain function for ethical meat and tissue production can therefore reduce the environmental impact, such as greenhouse gas emissions and deforestation.

### 5. BRIEF DESCRIPTION OF THE DRAWINGS

These and other features, aspects, and advantages of the present invention will become better understood with regard to the following description, and accompanying drawings, where:

FIG. 1A-1D presents a histological comparison between a genetically modified embryo and its wild type (WT) counterpart from the same litter, both of which were implanted simultaneously in the recipient female. FIG. 1A depicts a cross-section of the whole brain of the genetically modified embryo where genes NDE1 and DCX have been inactivated using CRISPR technology. FIG. 1B depicts a cross-section of the cortex of the genetically modified embryo where genes NDE1 and DCX have been inactivated using CRISPR technology. FIG. 1C depicts a cross-section of the whole brain of the WT embryo. FIG. 1D depicts a cross-section of the cortex of the WT embryo.

FIG. 2 provides a complete view of the embryo which underwent CRISPR-mediated genetic modification targeting the NDE1 and DCX genes, as at embryonic day 13 (E14).

FIG. 3A-3H is an immunohistochemical comparison between a genetically modified embryo with targeted knockouts of IL1RAPL1, CHRNA7, and their wild type counterpart. FIG. 3A displays the whole E14 mouse brain from a wild type embryo, stained for IL1RAPL1. The diffuse darker staining throughout the brain indicates a global expression of IL1RAPL1. FIG. 3B is an enlarged view of the cortex from the same wild type brain as in Frame A. Here, the surface of the cortex shows even darker staining, denoting higher IL1RAPL1 expression, as highlighted by arrows. FIG. 3C shows the whole brain of the knockout embryo, stained for IL1RAPL1. The brain appears much lighter, indicating a lack of IL1RAPL1 expression. Additionally, dysmorphic features and enlarged ventricles are evident, along with a noticeable reduction in brain matter. FIG. 3D provides an enlarged view of the cortex from the knockout embryo in Frame C. This frame shows no staining for IL1RAPL1, aligning with the knockout of this gene. FIG. 3E features another section of the same wild type brain from Frames A and B, but stained for CHRNA7. This frame shows diffuse CHRNA7 presence, with several regions displaying more concentrated expression, as indicated by arrows. FIG. 3F displays an enlargement of the cortex from the same wild type brain as in Frame E. The outer cortex exhibits clear dark staining, indicating high levels of CHRNA7 expression. FIG. 3G displays another section of the same knockout brain as in Frames C and D, stained for CHRNA7. The brain section replicates the dysmorphic features of Frame C and shows no CHRNA7 staining. FIG. 3H displays an enlargement of the cortex from the knockout brain in Frame G, demonstrating a complete absence of CHRNA7 staining.

FIG. 4 illustrates the expected additivity from multiplexing various mutations on the phenotypic reduction of brain tissue in the engineered organisms of the present disclosure.

FIG. 5 illustrates an engineered animal transitioning at an ideal time from natural womb to artificial support systems.

FIG. 6 shows knockout status of 3 different mouse fetal specimens treated with CRISPRs targeting GRIN2b and MFSD2a. All fetuses were alive and developing up to the point of measurement. Lane 0 is the control, specimen 1 was ran in lanes 1 and 2, specimen 2 was ran in lanes 3 and 4, specimen 3 was ran in lanes 5 and 6.

FIG. 7 shows knockout status of two genes, NDE1 (N) and MFSD2a (M) via excision, in 5 different fetal specimens. All fetuses were alive and developing up to the point of measurement. N1 and M1 show the homozygous wildtype form for each gene. Shorter bands for N and M display knockout conditions.

FIG. 8 shows knockout status of 3 different fetal specimens treated with CRISPRs targeting CHRNB2 and IL1RAPL1. Specimen G1-01 is homozygous for wild-type genes and was ran in lanes 1 and 2, specimen G1-04 is homozygous for knockout genes and was ran in lanes 3 and 4, specimen G1-02 was ran in lanes 5 and 6.

### 6. DETAILED DESCRIPTION OF THE INVENTION

### 6.1. Genetically Engineered Livestock

In the first aspect, genetically engineered organisms are provided. The genomic modifications prevent higher brain development during embryonic and fetal maturation.

### 6.1.1. Genomic Modifications to Prevent Higher Brain Development

In typical embodiments, mutations are not engineered into genes for which spontaneous mutations causing complete or near-complete anencephaly have been identified; in typical embodiments, the organisms are wild-type at these loci. These genes are known to broadly impact the functioning of centrioles, centromeres, microtubules or cell cycle checkpoint proteins (e.g., CDK6, MCPH1, CENPJ, and WDR62). Targeting such broadly expressed factors to prevent brain formation could lead to incomplete or improper development, subsequent degeneration, and/or altered function of the musculoskeletal system and other peripheral organs, limiting the utility of the organisms for either meat or organ production. In addition, the anencephalic phenotype caused by mutations in such genes is sporadic, with significant variation in phenotypic outcome (i.e., resulting brain development) from the same single gene alterations.

Therefore, in typical embodiments, a more controlled and specific genomic modification strategy is employed. In various embodiments, genes targeted for modification are largely specific to neural tissue or have highly enriched expression in neural tissue, and have a limited impact on non-neural tissue.

Naturally occurring mutations and animal models show that a spectrum of reduced brain developmental states are possible in addition to full deletion of the cerebrum that occurs with neural tube closure failures resulting in anencephaly. Whereas neural tube closure failures are typically multifactorial and do not occur with reliability, certain genetic mutations that can cause reduced brain development have consistent outcomes in humans and in animals. In some embodiments, the gene is selected from CIT, DCX, FTCD, GRIK3, GRIN2A, GRIN2B, MFSD2A, NDE1, NTRK1, PAFAH1B1 (LIS1), RELN, and TUBB3, or any established “microcephaly primary hereditary” (MCPH) genes (tier 1 genes).

While individual mutations are reproducible, they are unlikely to fully restrict higher brain formation on their own. Thus, in some embodiments, modifications are made to a plurality of genes. This multiplexing enables strategic combinations of genetic alterations to be deployed to further limit brain development and neuron survival, causing deletion of all, or of components, of the cerebrum. Using combinations of these key gene deletions with other gene deletions or other interventions ensures the profound and reliable prevention of higher nervous system formation.

In some embodiments in which a plurality of genes are modified, at least one of the plurality of genes to be modified is selected from CIT, DCX, FTCD, GRIK3, GRIN2A, GRIN2B, MFSD2A, NDE1, NTRK1, PAFAHIB1 (LIS1), RELN, and TUBB3, or any established MCPH gene (tier 1).

In certain embodiments in which a plurality of genes are modified, at least one of the plurality is a gene that affects neuronal signaling, preventing transmission or perception of pain signals. Modifying genes that alter the perception of pain or the ability to feel pain is beneficial, either on its own or in combination with other gene alterations that limit neural development. In some embodiments, such a gene is selected from NTRK1, PRDM12, genes related to monoamine neurotransmitter synthesis and production and serotonergic or dopaminergic neuron development or function.

Genes for the 5HT receptors (including HTR6, GPR26), dopamine receptors (DRD3), GABA receptors (GABRA6), glutamate receptors (GRM2, GRM4, SLC1A2), Interleukin-1 receptor family (IL1RAPL1), transcription factors (such as SOX1, TBR1, VAX1) potassium channels (KCNK4), and PRDM12 (tier 2) are also usefully modified in combination gene modification strategies.

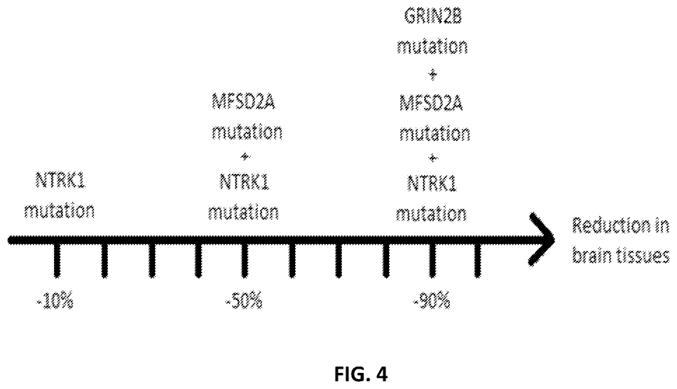

One such combination is shown in FIG. 4. In some embodiments, modifications are made in a plurality of genes selected from NTRK1, MFSD2A and GRIN2B. NTRK1 can be altered to prevent any sensation of pain. Certain DNA alterations in NTRK1 can also result in a progressive neuron loss that is significant enough to affect global brain function. Additionally, these mutations may also sensitize surviving neurons to apoptosis, leading to an increase in efficacy of further intentional or unintentional stressors to cause prevention of development of neural mass. MFSD2A can be altered to inhibit proper transport of certain fatty acids such as docosahexaenoic acid (DHA), thereby preventing proper neuron formation, which results in significantly reduced brain development. GRIN2B (NMDA receptor) alteration results in neuron specific effects that can range from minor to major developmental impairment. The specific altered form of GRIN2B used can depend on the most favorable synergistic effect achieved through experimentation.

In some embodiments, modifications are made to genes that regulate stem cell maintenance and expansion, and/or genes that are otherwise related to the formation and proper function of neurons and their support cells.

The gene modifications can be performed on germ cells, fertilized eggs, early stage embryos, or any other cell type that can be induced to revert to an embryonic-like state.

In some embodiments, genetic modification reduces expression of the encoded protein. In certain embodiments, the modification is a complete knock-out, eliminating expression of the encoded protein.

In some embodiments, the modification is introduction of an indel into the coding sequence, leading to frameshift and truncation, with the truncated protein having reduced function, no function, and/or reduced functional half-life. In certain embodiments, indels are introduced by nonhomologous end joining following a double strand break affected by an RNA guided DNA nuclease, such as CRISPR-Cas9.

In some embodiments, the modification alters the primary amino acid sequence of the protein, either by insertion of a modified gene or by editing the endogenous gene, such as by PRIME editing. In certain embodiments, the sequence of the protein is mutated in order to cause it to aggregate, or specific amino acid sequences can be added or removed to induce misfolding and aggregation of the protein. The mutation of the protein's sequence or the induction of misfolding and aggregation through the addition or removal of specific amino acid sequences can contribute to cellular stress and apoptosis. These changes to the protein can disrupt normal cellular processes and lead to the death of the cell.

Manipulations can include insertions, deletions, or other modifications to the gene(s) of interest or the corresponding regulatory elements. Modifications can be homozygous or heterozygous.

### 6.1.2. Further Genomic Modifications for Xenotransplantation Organ Production

In some embodiments, engineered livestock are created to provide organs for xenotransplantation. In certain of these embodiments, further genomic modifications can be made to reduce immunogenicity following xenogeneic transplantation into a human host.

In certain embodiments, the cells are porcine and the further engineering is a knock-out of at least one gene selected from GGTA1, CMAH, B5GALNT2, β2M, and CIITA. In some embodiments, the further engineering is knock-out of a plurality of genes selected from GGTA1, CMAH, B5GALNT2, β2M, and CIITA. In some embodiments, the animal's cells are further engineered to express at least one human transgene selected from CD39, CD46, CD47, CD55, EPCR, TFPI, THBD, HO-1, vWF, and HLA-E.

In particular embodiments, the tissue to be transplanted is cartilage, cardiac valve, heart, kidney, pancreatic islets, or lung.

### 6.1.3. Gestational Maturation

In one series of embodiments, the genetically modified embryo is implanted into a surrogate's uterus, where it receives nourishment and support as it grows and develops. This embodiment allows for the natural development of the genetically modified embryo within the womb, taking advantage of the benefits provided by the maternal environment. Life support is provided following natural or induced birth.

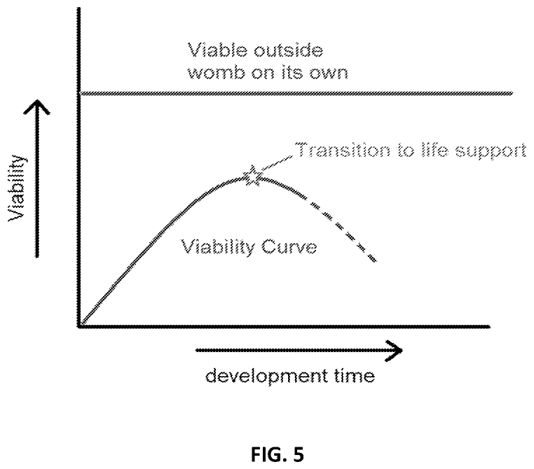

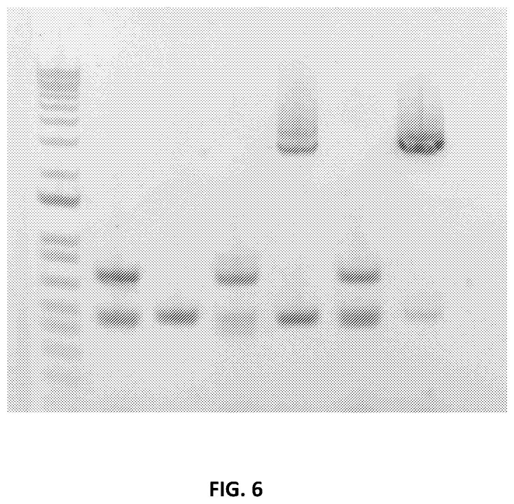

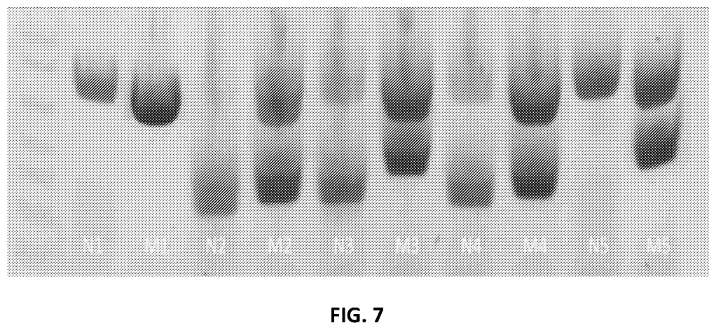

As is true in natural cases of pregnancy where the fetus lacks the majority of its brain, the fetus may not be viable long enough to carry to term in a surrogate pregnancy. In various embodiments, engineered organisms, in particular those with multiple genetic alterations to limit brain development, will have a greater need for support, and at an earlier stage of development. Transition to support systems may have to occur before viability due to insufficient brain and spinal cord development. FIG. 5 illustrates an engineered animal transitioning at an ideal time from natural womb to artificial support systems.

In some embodiments, the development of neural tissue can be monitored using methods such as ultrasound or other means.

In some embodiments, modifications are additionally made to genes that cause neurons to be more susceptible to apoptosis in response to changes in temperature, nutrient availability, oxygen availability, small molecule inhibitors, or other toxin exposures, compared to normally functioning neurons. These conditions can be adjusted during embryonic and fetal maturation to further reduce the development and survival of nervous tissue.

In some embodiments, factors that enhance the stability or resistance of developing tissues to stressors or apoptosis are prophylactically introduced or in response to changing parameters. These may include antioxidants, anti-apoptotic small molecules, and growth factors. An automatic sampling and monitoring system can be implemented to precisely adjust interventions in response to toxin levels, nutrient uptake, the state of nervous tissue, and basic vital signs.

### 6.2. Engineered Tissues

In another aspect, genetically engineered tissues are provided. The tissues comprise a plurality of cells cohered into a three-dimensional structure, wherein the cells have differentiated from a single zygote into at least two differentiated cell types, and wherein the cells commonly contain at least one genomic alteration that is neuronal signal reducing, neuron-depleting and neuron disrupting. In some embodiments, the cells contain a plurality of genomic alterations that are collectively in effect, at least one of the following neuronal signal reducing, neuron-depleting and neuron disrupting.

In some embodiments, the cells are mammalian cells. In certain embodiments, the cells contain at least one genomic alteration that reduces expression of at least one gene selected from CIT, DCX, FTCD, GRIK3, GRIN2A, GRIN2B, MFSD2A, NDE1, NTRK1, PAFAH1B1 (LIS1), RELN, and TUBB3. In some embodiments, the cells contain at least one genomic alteration that reduces function of the protein respectively encoded by at least one gene selected from CIT, DCX, FTCD, GRIK3, GRIN2A, GRIN2B, MFSD2A, NDE1, NTRK1, PAFAHIB1 (LIS1), RELN, and TUBB3.

In certain embodiments, the cells contain genomic alterations in MFSD2A and NDE1.

In some embodiments, the genomic alteration comprises one or more mutations to the primary amino acid sequence of the protein. In particular embodiments, the one or more mutations cause misfolding and aggregation of the protein.

In some embodiments, the cells further contain at least one genomic alteration that reduces expression of at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

In some embodiments, the cells further contain at least one genomic alteration that reduces function of the protein respectively encoded by at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

In particular embodiments, the genomic alteration comprises one or more mutations to the primary amino acid sequence of the further altered protein. In certain embodiments, the one or more mutations cause misfolding and aggregation of the protein.

In some embodiments, the cells are homozygous for at least one of the described genomic alterations. In some embodiments, the cells are heterozygous for at least one of the described genomic alterations. In some embodiments, the tissue is in utero. In some embodiments, the tissue is ex vivo.

In some embodiments, the mammalian cells are from Family Bovidae or Family Suidae.

### 6.2.1. Organs for Transplantation

In some embodiments, the cells of the tissue have been further engineered to reduce immunogenicity following transplantation into a human host. In certain embodiments, the further engineering is a knock-out at least one gene selected from GGTA1, CMAH, B5GALNT2, β2M, and CIITA. In certain embodiments, the further engineering is knock-out of a plurality of genes selected from GGTA1, CMAH, B5GALNT2, β2M, and CIITA. In some embodiments, the cells are porcine.

In some embodiments, the cells of the tissue have been further engineered to express at least one human transgene selected from CD39, CD46, CD47, CD55, EPCR, TFPI, THBD, HO-1, vWF, and HLA-E.

In some embodiments, the tissue is cartilage, cardiac valve, heart, kidney, pancreatic islets, or lung.

### 6.3. Definitions

Unless defined otherwise, all technical and scientific terms used herein have the meaning commonly understood by a person skilled in the art to which this invention belongs. The genes selected herein are defined as: being interchangeably the selected gene and any known gene(s) or its protein product with functional equivalency and/or a high degree of regional or gene/protein wide sequence homology that would have a reasonable expectation of a similar outcome to the selected gene, in context of its use.

### 6.4. Additional Embodiments

In one aspect, provided is a genetically engineered tissue, comprising a plurality of cells cohered into a three-dimensional structure, wherein the cells have differentiated from a single zygote into a plurality of differentiated cell types, and wherein the cells commonly contain at least one genomic alteration that is at least one of the following neuronal signal reducing, neuron-depleting and neuron disrupting. In various embodiments, the cells contain a plurality of genomic alterations that are collectively in effect that is at least one of the following neuronal signal reducing, neuron-depleting and neuron disrupting. In some embodiments of the genetically engineered tissue, the cells are mammalian cells.

In various embodiments of the genetically engineered tissue, the cells contain at least one genomic alteration that reduces expression of at least one gene selected from CIT, DCX, FTCD, GRIK3, GRIN2A, GRIN2B, MFSD2A, NDE1, NTRK1, PAFAHIB1 (LIS1), RELN, and TUBB3. In some embodiments, the cells contain at least one genomic alteration that reduces function of at least one protein respectively encoded by a gene selected from CIT, DCX, FTCD, GRIK3, GRIN2A, GRIN2B, MFSD2A, NDE1, NTRK1, PAFAHIB1 (LIS1), RELN, and TUBB3. In some embodiments, the genomic alteration comprises one or more mutations to the primary amino acid sequence of the protein. In some embodiments, the one or more mutations cause misfolding and aggregation of the protein. In some embodiments, the cells contain genomic alterations in MFSD2A and NDE1.

In some embodiments of the genetically engineered tissue, the cells further contain at least one genomic alteration that reduces expression of at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

In some embodiments of the genetically engineered tissue, the cells further contain at least one genomic alteration that reduces function of the protein respectively encoded by at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

In some further embodiments of the genetically engineered tissue, the genomic alteration comprises one or more mutations to the primary amino acid sequence of the further altered protein. In some embodiments, the one or more mutations cause misfolding and aggregation of the protein.

In various embodiments of the genetically engineered tissue, the cells are homozygous for at least one genomic alteration. In some embodiments, the cells are heterozygous for at least one genomic alteration.

In some embodiments, the mammalian cells are from Family Bovidae or Family Suidae.

In some embodiments, the tissue is in utero. In some embodiments, the tissue is ex vivo.

In some embodiments, the cells are from a species of cattle, sheep, goats, pigs or rabbits.

In some embodiments of the genetically engineered tissue, the tissue is non-human and the cells have been further engineered to reduce immunogenicity following xenogeneic transplantation into a human host. In some embodiments, the cells are porcine and the further engineering is a knock-out of at least one gene selected from GGTA1, CMAH, B5GALNT2, β2M, and CIITA. In some embodiments, the further engineering is knock-out of a plurality of genes selected from GGTA1, CMAH, B5GALNT2, β2M, and CIITA. In some embodiments, the cells are further engineered to express at least one human transgene selected from CD39, CD46, CD47, CD55, EPCR, TFPI, THBD, HO-1, vWF, and HLA-E. In some embodiments, the tissue is cartilage, cardiac valve, heart, kidney, pancreatic islets, or lung.

In another aspect, provided are methods of producing a genetically engineered tissue comprising a plurality of cells cohered into a three dimensional structure, wherein the cells have differentiated from a single zygote into at least two differentiated cell types, and wherein the cells commonly contain at least one genomic alteration is at least one of the following neuronal signal reducing, neuron-depleting and neuron disrupting, where the method comprises preparing an embryo ex vivo in which the cells commonly contain at least one genomic alteration that is at least one of the following neuronal signal reducing, neuron-depleting and neuron disrupting, and implanting the embryo into the uterus of a suitably prepared host surrogate.

In some embodiments of the method, the method further comprising the subsequent step, after a period of in utero growth and maturation that is less than the full gestational period, of transferring the embryo from host uterus to artificial life support. In some embodiments, after a full gestation in utero, the method further comprises of delivering the full-term animal.

In various embodiments of the method, the method further comprising the later step of sacrificing the animal and harvesting the tissue.

In another aspect, provided is an engineered organism, comprising cells having at least one genomic alteration that is at least one of the following neuronal signal reducing, neuron reducing, neuron-depleting and neuron disrupting. In various embodiments, the cells of the engineered organism contain a plurality of genomic alterations that are collectively in effect at least one of the following neuronal signal reducing, neuron reducing, neuron-depleting and neuron disrupting.

In various embodiments of the engineered organism, the organism's cells contain at least one genomic alteration that reduces expression of at least one gene selected from CIT, DCX, FTCD, GRIK3, GRIN2A, GRIN2B, MFSD2A, NDE1, NTRK1, PAFAHIB1 (LIS1), RELN, and TUBB3.

In various embodiments of the engineered organism, the cells contain at least one genomic alteration that reduces function of at least one protein respectively encoded by a gene selected from CIT, DCX, FTCD, GRIK3, GRIN2A, GRIN2B, MFSD2A, NDE1, NTRK1, PAFAHIB1 (LIS1), RELN, and TUBB3.

In various embodiments of the engineered organism, the organism's cells further contain at least one genomic alteration that reduces expression of at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

In various embodiments of the engineered organism, the cells further contain at least one genomic alteration that reduces function of the protein respectively encoded by at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

In various embodiments of the engineered organism, the organism's cells contain at least one genomic alteration that reduces expression of at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

In various embodiments of the engineered organism, the cells contain at least one genomic alteration that reduces function of the protein respectively encoded by at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

In various embodiments of the engineered organism, the cells contain genomic alterations in MFSD2A and NDE1.

### 6.5. Other Interpretational Conventions

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

In this disclosure, “comprises”, “comprising”, “containing”, “having”, “includes”, “including” and linguistic variants thereof have the meaning ascribed to them in U.S. Patent law, permitting the presence of additional components beyond those explicitly recited.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art. Unless otherwise specified, “about” intends ±10% of the stated value. Where a percentage is provided with respect to an amount of a component or material in a composition, the percentage should be understood to be a percentage based on weight, unless otherwise stated or understood from the context.

### 7. EXAMPLES

### 7.1. Zygote Genome Editing

This example demonstrates the methodology at the which the successful editing of select genes in zygotes using Cas9 nuclease system was performed. Protospacer adjacent motifs (PAM) site recognition by Cas9 nucleases is notably essential for the exploitation as effective genome editing tools and are provided as well as guide RNA (gRNA) target, gRNA sequences, and polymerase chain reaction (PCR) primers used in the exploitation of Cas9 nuclease system for each gene target. Tables 1-42.

Methods and Materials: guide RNAs (gRNAs) were incubated together in various combinations for a minimum of 30 minutes with CAS9 in OPTI-MEM. The resulting ribonucleoparticles (RNPs) were electroporated into mouse zygotes at their pronuclear stage using an ECM830 square electroporator. Typically, this was performed at a voltage of 30V, using 5 pulses, a pulse length of 3 minutes, at an interval of 100 milliseconds (ms).

### Gene: NDE

TABLE 1
gRNA target sequences (PAM underlined)

| Direction | Sequence | SEQ ID NO: |
|---|---|---|
| matching reverse | TGTTTCCTCATC | 1 |
| strand of gene | TCACGGTAAGG | |
| matching forward | AATGTACAGTAG | 2 |
| strand of gene | TTCTCCTTCGG | |
| matching reverse | TCACAGCTGCTG | 3 |
| strand of gene | CCTTAAGCAGG | |
| matching reverse | CCCAGCACTCAA | 4 |
| strand of gene | AGGACCGAAGG | |

TABLE 2
gRNA sequence

| Sequence | SEQ ID NO: |
|---|---|
| UGUUUCCUCAUCUCACGGUA | 5 |
| AAUGUACAGUAGUUCUCCUU | 6 |
| UCACAGCUGCUGCCUUAAGC | 7 |
| CCCAGCACUCAAAGGACCGA | 8 |

TABLE 3
PCR Primers

| Direction | Sequence | SEQ ID NO: |
|---|---|---|
| Forward primer (F1) | 5′-TTTTCTCCTAGA | 9 |
| | CATCATGCCACA-3′ | |
| Reverse primer (R1) | 5′-CCAGTGGAACAA | 10 |
| | GTACTGAAAGCTA-3′ | |

| | |
|---|---|
| Targeted allele | 335 bp |
| Wildtype allele | 20048 bp |

### Gene: MFSD2a

TABLE 4
gRNA target sequences (PAM underlined)

| | | SEQ ID |
|---|---|---|
| Direction | Sequence | NO: |
| matching reverse strand of gene | CAACGTCGTATCTCCGATAACGG | 11 |
| matching reverse strand of gene | CTAACTAAGCCATACCATACTGG | 12 |
| matching forward strand of gene | ATCATCACCACGATGTTGTTGGG | 13 |
| matching reverse strand of gene | CCAGCAGGGCATCAGAATACAGG | 14 |

TABLE 5
gRNA sequence

| Sequence | SEQ ID NO: |
|---|---|
| CAACGUCGUAUCUCCGAUAA | 15 |
| CUAACUAAGCCAUACCAUAC | 16 |
| AUCAUCACCACGAUGUUGUU | 17 |
| CCAGCAGGGCAUCAGAAUAC | 18 |

TABLE 6
PCR Primers

| Direction | Sequence | SEQ ID NO: |
|---|---|---|
| Forward primer (F1) | 5′-AACACACTGTAGCTGATACGTCTT-3′ | 19 |
| Reverse primer (R1) | 5′-CTAAACCACAGGACAGTTTACTCAC-3′ | 20 |
| Targeted allele | 483 bp | |
| Wildtype allele | 10267 bp | |

### Gene: GRIN2B

TABLE 7
gRNA target sequences (PAM underlined)

| | | SEQ ID |
|---|---|---|
| Direction | Sequence | NO: |
| matching reverse strand of gene | GAAGTTGAAACCTGGTATTTTGG | 21 |
| matching reverse strand of gene | CCACTACAGCAACTTTCCAAAGG | 22 |

TABLE 8
gRNA sequence

| Sequence | SEQ ID NO: |
|---|---|
| GAAGUUGAAACCUGGUAUUU | 23 |
| CCACUACAGCAACUUUCCAA | 24 |

TABLE 9
PCR Primers

| Direction | Sequence | SEQ ID NO: |
|---|---|---|
| Forward primer (F1) | 5′-GCTTAGAGGTCCAATTTCTTGGTCT-3′ | 25 |
| Reverse primer (R1) | 5′-GTTGTTCCCTTCTAGTGTTTTCTCG-3′ | 26 |
| Targeted allele | 478 bp | |
| Wildtype allele | 2761 bp | |

### Gene: DCX

TABLE 10
gRNA target sequences (PAM underlined)

| | | SEQ ID |
|---|---|---|
| Direction | Sequence | NO: |
| matching forward strand of gene | GGGCAGCCAAAAGCATCTGATGG | 27 |
| matching forward strand of gene | TATACAGCATGATGCAACCTTGG | 28 |

TABLE 11
gRNA sequence

| Sequence | SEQ ID NO: |
|---|---|
| GGGCAGCCAAAAGCAUCUGA | 29 |
| UAUACAGCAUGAUGCAACCU | 30 |

TABLE 12
PCR Primers

| Direction | Sequence | SEQ ID NO: |
|---|---|---|
| Forward primer (F1) | 5′-ACCTTGAGGCTACTATGCTTTACTC-3′ | 31 |
| Reverse primer (R1) | 5′-TCAATCCAAATGGATGCAGAACAC-3′ | 32 |
| Targeted allele | 391 bp | |
| Wildtype allele | 2187 bp | |

### Gene: I11RAPL1

TABLE 13
gRNA target sequences (PAM underlined)

| | | SEQ ID |
|---|---|---|
| Direction | Sequence | NO: |
| matching reverse strand of gene | AAAAGAGTACCTGATGACACAGG | 45 |
| matching forward strand of gene | CAGCTGATGGATGCACAGATTGG | 46 |

TABLE 14
gRNA sequence

| Sequence | SEQ ID NO: |
|---|---|
| AAAAGAGUACCUGAUGACAC | 47 |
| CAGCUGAUGGAUGCACAGAU | 48 |

TABLE 15
PCR Primers

| Direction | Sequence | SEQ ID NO: |
|---|---|---|
| Forward primer (F1) | 5′-ACATGGCAACACATGCAAATA-3′ | 49 |
| Reverse primer (R1) | 5′-CTGACTCAATACATATGCTCTGTAAAC-3′ | 50 |
| Targeted allele | 254 bp | |
| Wildtype allele | 513 bp | |

### Gene: CHRNB2

TABLE 16
gRNA target sequences (PAM underlined)

| | | SEQ ID |
|---|---|---|
| Direction | Sequence | NO: |
| matching forward strand of gene | ATTGGCACAGCTCATCAGTGTGG | 87 |
| matching reverse strand of gene | GGTTTTGGGTACTGACACAGAGG | 88 |

TABLE 17
gRNA sequence

| Sequence | SEQ ID NO: |
|---|---|
| AUUGGCACAGCUCAUCAGUG | 89 |
| GGUUUUGGGUACUGACACAG | 90 |

TABLE 18
PCR Primers

| Direction | Sequence | SEQ ID NO: |
|---|---|---|
| Forward primer (F1) | 5′-CAGGGCAGCTGCTGAAA-3′ | 91 |
| Reverse primer (R1) | 5′-AAGACAATCGGCCAACTCTG-3′ | 92 |
| Targeted allele | 335 bp | |
| Wildtype allele | 458 bp | |

### 7.3. Confirmation of Gene Knockouts (KO)

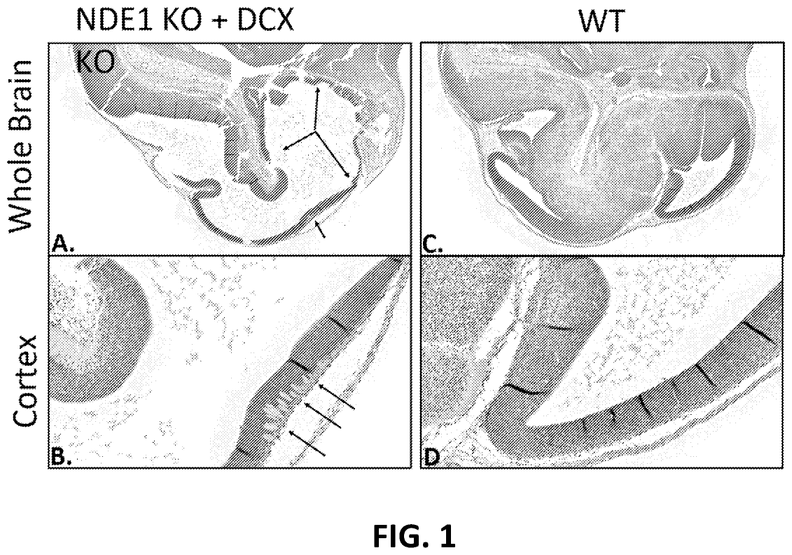

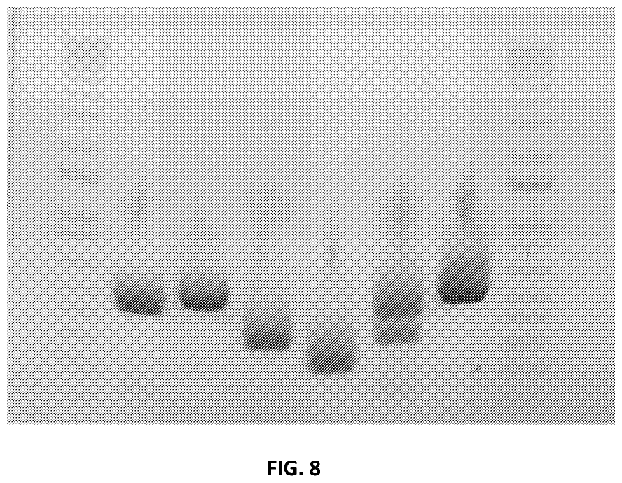

Gene knockouts were subsequently confirmed via histological studies (FIGS. 1-3) and PCR results (FIG. 6-8)

### Comparative Histological Analysis of Genetically Modified and Wild Type Embryonic Brains

The results displayed in FIG. 1A-1D presented a histological comparison between a genetically modified embryo and its wild type counterpart from the same litter, both of which were implanted simultaneously in the recipient female. The left panels depicted the genetically modified embryo, while the right panels featured the wild type embryo. More specifically, FIG. 1A showcased the whole brain of the genetically modified embryo, where genes NDE1 and DCX had been inactivated using CRISPR technology. This panel highlighted significant morphological differences, including dysmorphic features and areas of active degeneration in the cortex and other brain regions, as indicated by arrows. Notably, there was a pronounced enlargement of the ventricles and a developmental abnormality characterized by the ventricles' failure to separate properly at this developmental stage. The cerebellum did not appear to be in the process of formation at all. FIG. 1B showed a magnified view of the cerebral cortex in the genetically modified embryo. This detailed view underscored the extent of cortical anomalies and degenerative changes, and further complemented the observations of ventricular enlargement and malformation. FIG. 1C showed the whole brain of the wild type sibling embryo at a similar developmental stage for comparison. The morphology, particularly the ventricular structure and overall brain architecture, contrasted sharply with that of the genetically modified embryo, where enlarged ventricles and a failure of ventricular separation were observed. FIG. 1D provided an enlarged view of the cerebral cortex in the wild type embryo, illustrating normal cortical development. This panel served as a control, demonstrating the typical cortical structure absent in the genetically modified counterpart.

### Whole Embryo Visualization of the Genetically Modified Embryo at Embryonic Day 14 (E14)

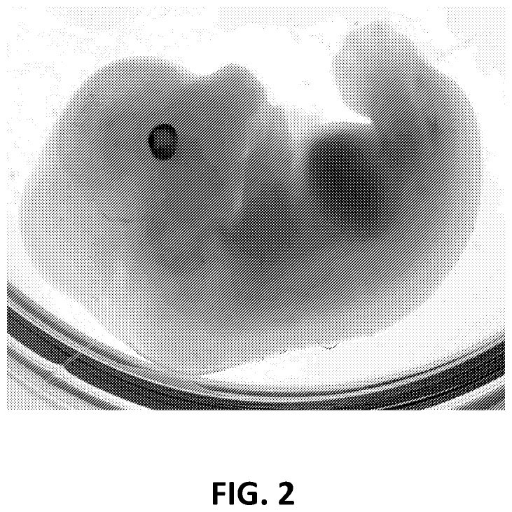

The results displayed in FIG. 2 captured the entire embryo at embryonic day 14 (E14). Notably, despite the pronounced cerebral modifications as detailed above and in FIG. 1A-1D, the external morphology of the embryo appears unremarkable and is consistent with typical developmental milestones for this stage. A key finding at the time of harvest was the presence of an active heartbeat. This indicated embryonic vitality. This observation was significant as it highlighted the localized nature of the cerebral changes, with no evident impact on the embryo's overall viability.

It is important to emphasize that the genetic modifications primarily affected the brain development, as extensively analyzed in FIG. 1A-1D. This figure underscored the contrast between the extensive brain abnormalities and the otherwise normal external development of the embryo. It illustrated that profound internal changes in brain morphology may not be externally visible at this developmental stage.

Notably, despite significant cerebral abnormalities in the genetically modified embryo, its overall embryonic development was grossly normal including an active heartbeat and the unremarkable appearance of other major organs at the time of harvest. FIG. 2.

### Detailed Immunohistochemical Analysis of ILIRAPL1 and CHRNA7 in Genetically Modified and Wild Type E14 Mouse Brains

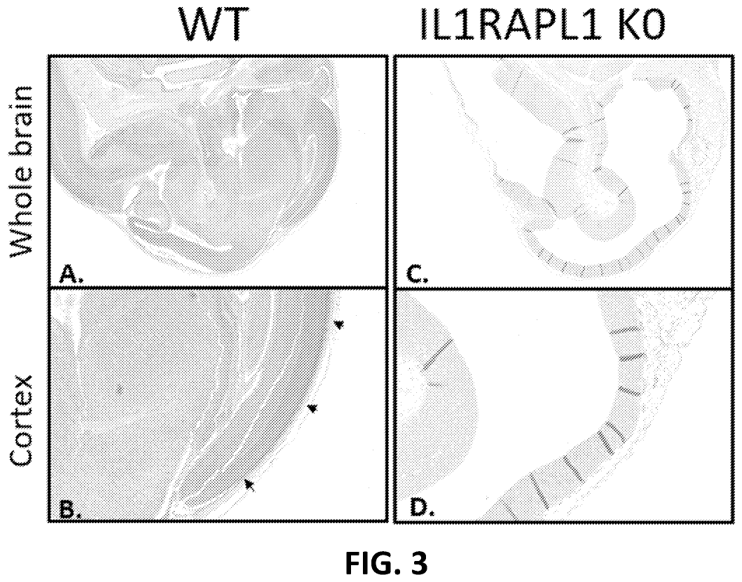

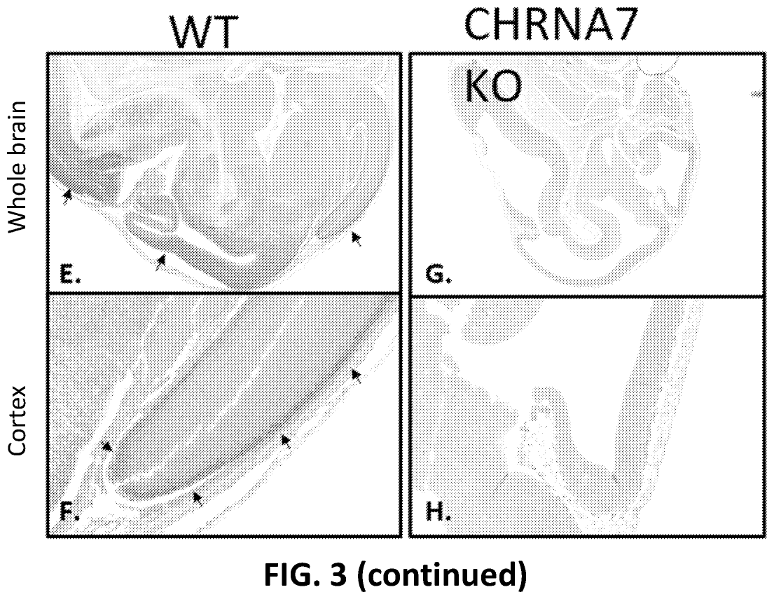

Results indicated that the immunohistochemical comparison between a genetically modified embryo with targeted knockouts of IL1RAPL1 and CHRNA7. FIG. 3 underscored the significant impact of IL1RAPL1, CHRNB2 and CHRNA7 knockouts on brain morphology and function. FIG. 3A-3H. The combination of these 3 knockouts significantly and substantially altered brain structure. Basic brain structure and development was compromised. Acetylcholine signaling was also disrupted. As a result, brain function and cognition was expected to be profoundly affected, other tissues and organ development were not significantly compromised.

PCR results show that multiple combinations of knockouts can still result in the production of fetuses with the potential to develop to late term (FIG. 6-8). Histological results show both expression deficits and structural abnormalities in animals confirmed with knockout variants of the targeted genes.

### 7.4. Implantation of KO Embryos in Recipient Targets

The resulting knockout (KO) embryos were group cultured in groups sizing between 20 and 120 for 72 to 96 hours, in Embryomax® KSOM culture medium or similarly supportive mouse embryo culture media under mineral oil in an incubator at 37.0° C. and 5% CO². Conditions were adjusted to maintain a pH between 7.2 and 7.4.

Embryos at the blastocyst stage were transferred into roughly 2.5DPC pseudopregnant female CD1 (ARC) mice with an implantation target weight of approximately 30 g. Recipient mouse target age was >8 full weeks of age. Synchronization was induced with either 2.5IU pregnant mare serum gonadotropin (PMSG) followed by 2.5IU-5IU human chorionic gonadotropin (HCG) 48 hours later or 50 μl hyperova followed by 5IU HCG 48 hours later. Approximately 7 hours following HCG administration, pseudopregnancy was induced in properly cycled females via mechanical and vibration-based stimulation by inserting a smooth plastic rod into the vagina for 30 seconds, which could be contacted with an electric toothbrush module or trimmer.

The embryos were then transferred directly into the uterus of the recipient females at the receptive stage 2.5 days later directly through the vagina, non-surgically, using a nonsurgical embryo transfer (NSET) device (Paratechs). FIG. 6 and FIG. 7 represent the methods used to process the histological images referenced below. All wild type and knockout embryos compared with each other underwent identical processing.

### 7.5. Implications of Combined Genetic Knockouts on Cognition in Animal Models

The data presented in FIG. 1A-3H offered compelling evidence of the profound effects of specific genetic knockouts on brain development and, by extension, on overall cognition in mice. These figures collectively demonstrated that the combined knockouts of brain-related and specific genes had more significant impacts than single-gene alterations.

In FIG. 1A-1D, we observed the cerebral changes in embryos with CRISPR-mediated knockouts of NDE1 and DCX genes. The dysmorphology of the brain, particularly the enlargement of ventricles and the failure of ventricular separation, pointed towards significant developmental disruptions. These structural anomalies, especially in regions critical for cognitive processes, suggested a potential for substantial cognitive deficits in these animals.

FIG. 2 complemented these findings by showing that, despite profound internal brain changes, the overall embryonic development appeared grossly normal. This observation was critical as it underscored the specificity of genetic modifications in affecting brain development while leaving other developmental processes relatively unaffected. Organs and tissues remained functional and intact.

FIG. 3A-3H illustrated the effects of knocking out ILIRAPL1 and CHRNA7. The absence of IL1RAPL1 and CHRNA7 expression in the knockout models, as evidenced by the distinct staining patterns, highlighted the crucial roles these genes play in normal brain function. Given the known associations of ILIRAPL1 with mental retardation and CHRNA7 with neuronal acetylcholine receptor function, their absence strongly indicate potential impairments in cognitive abilities. The failure of CHRNB2 staining, while not visually documented, added another layer to our understanding of the complex gene interactions influencing brain development.

Collectively, these results suggested that the combined effect of multiple gene knockouts on brain morphology and function is greater than the sum of individual knockouts. This synergistic impact was particularly evident in the areas critical for cognition, implying that animals with these genetic modifications were likely to experience significant and profound cognitive deficiencies.

### 7.6. Organ Development in KO Mice Fetus

In our exploration of the developmental impacts of genetic modifications, a particularly intriguing observation was the grossly normal development of organs and tissues, despite significant aberrations in brain morphology and function. FIG. 2 demonstrated this phenomenon, showcasing an E14 embryo with a distinct active heartbeat, an indicator of overall embryonic vitality, despite the profound cerebral abnormalities induced by genetic knockouts, as detailed in FIG. 1A-1B of this animal. This finding was remarkable in that it suggested a degree of developmental compartmentalization, where severe genetic alterations could lead to specific brain malformations without disrupting the general developmental trajectory of other organs and tissues. The presence of a normal heartbeat, alongside typical morphological features in major organs, reinforced this notion. It also raised intriguing questions about the resilience and adaptability of embryonic development in the face of targeted genetic disruptions, particularly those affecting brain development. Such observations, juxtaposing the brain changes and greatly retarded brain growth from FIGS. 1A-1D and FIGS. 3A-3H against the otherwise unremarkable overall development in FIG. 2, indicate complex interplay between genetic regulation, organ specificity, and developmental robustness.

### 8. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A genetically engineered tissue, comprising:
a plurality of cells cohered into a three-dimensional structure,
wherein the cells have differentiated from a single zygote into a plurality of differentiated cell types, and
wherein the cells commonly contain at least one genomic alteration that is at least one of the following neuronal signal reducing, neuron-depleting and neuron disrupting.

2. The tissue of claim 1, wherein the cells contain a plurality of genomic alterations that are collectively in effect at least one of the following neuronal signal reducing, neuron-depleting and neuron disrupting.

3. The tissue of claim 1 or claim 2, wherein the cells are mammalian cells.

4. The tissue of claim 3, wherein the cells contain at least one genomic alteration that reduces expression of at least one gene selected from CIT, DCX, FTCD, GRIK3, GRIN2A, GRIN2B, MFSD2A, NDE1, NTRK1, PAFAH1B1 (LIS1), RELN, and TUBB3.

5. The tissue of claim 3, wherein the cells contain at least one genomic alteration that reduces function of at least one protein respectively encoded by a gene selected from CIT, DCX, FTCD, GRIK3, GRIN2A, GRIN2B, MFSD2A, NDE1, NTRK1, PAFAHIB1 (LIS1), RELN, and TUBB3.

6. The tissue of claim 5, wherein the genomic alteration comprises one or more mutations to the primary amino acid sequence of the protein.

7. The tissue of claim 6, wherein the one or more mutations cause misfolding and aggregation of the protein.

8. The tissue of any one of claims 4-7, wherein the cells contain genomic alterations in MFSD2A and NDE1.

9. The tissue of any one of claims 4-8, wherein the cells further contain at least one genomic alteration that reduces expression of at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

10. The tissue of any one of claims 4-8, wherein the cells further contain at least one genomic alteration that reduces function of the protein respectively encoded by at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.

11. The tissue of claim 10, wherein the genomic alteration comprises one or more mutations to the primary amino acid sequence of the further altered protein.

12. The tissue of claim 11, wherein the one or more mutations cause misfolding and aggregation of the protein.

13. The tissue of any one of claims 1-12, wherein the cells are homozygous for at least one genomic alteration.

14. The tissue of any one of claims 1-12, wherein the cells are heterozygous for at least one genomic alteration.

15. The tissue of any one of claims 3-14, wherein the mammalian cells are from Family Bovidae or Family Suidae.

16. The tissue of any one of claims 3-15, wherein the tissue is in utero.

17. The tissue of any one of claims 3-15, wherein the tissue is ex vivo.

18. The tissue of claim 17, wherein the cells are from a species of cattle, sheep, goats, pigs or rabbits.

19. The non-human tissue of claim 15, wherein the cells have been further engineered to reduce immunogenicity following xenogeneic transplantation into a human host.

20. The non-human tissue of claim 19, wherein the cells are porcine and the further engineering is a knock-out of at least one gene selected from GGTA1, CMAH, B5GALNT2, B2M, and CIITA.

21. The non-human tissue of claim 20, wherein the further engineering is knock-out of a plurality of genes selected from GGTA1, CMAH, B5GALNT2, β2M, and CIITA.

22. The non-human tissue of claim 20 or claim 21, wherein cells are further engineered to express at least one human transgene selected from CD39, CD46, CD47, CD55, EPCR, TFPI, THBD, HO-1, vWF, and HLA-E.

23. The non-human tissue of any one of claims 19-22, wherein the tissue is cartilage, cardiac valve, heart, kidney, pancreatic islets, or lung.

24. A method of producing a genetically engineered tissue comprising a plurality of cells cohered into a three dimensional structure, wherein the cells have differentiated from a single zygote into at least two differentiated cell types, and wherein the cells commonly contain at least one genomic alteration that is at least one of the following neuronal signal reducing, neuron-depleting and neuron disrupting, the method comprising:
preparing an embryo ex vivo in which the cells commonly contain at least one genomic alteration that is at least one of neuronal signal reducing, neuron-depleting and neuron disrupting, and
implanting the embryo into the uterus of a suitably prepared host surrogate.

25. The method of claim 24, further comprising the subsequent step, after a period of in utero growth and maturation that is less than the full gestational period, of transferring the embryo from host uterus to artificial life support.

26. The method of claim 24, further comprising the subsequent step, after a full gestation in utero, of delivering the full term animal.

27. The method of claim 25 or claim 26, further comprising the later step of sacrificing the animal and harvesting the tissue.

28. An engineered organism, comprising: cells having at least one genomic alteration that is at least one of neuronal signal reducing, neuron-depleting and neuron disrupting.

29. The engineered organism of claim 28, wherein the organism's cells contain a plurality of genomic alterations that are collectively neuronal signal reducing, neuron-depleting and neuron disrupting.

30. The tissue of any one or combination of claim 4-10 or 28-29, wherein the tissue is generated for meat production and/or for the creation of organs for transplantation.

31. The organisms of any one or combination of claim 4-10 or 28-29 wherein they are engineered to have severely reduced ability to experience suffering or be totally incapable of suffering by lacking cognitive capability and/or pain reception through the use of a combination of listed genes.

32. The tissue of claim 30, wherein the cells contain more than one genomic alteration that reduces expression of at least one gene selected from CIT, DCX, FTCD, GRIK3, GRIN2A, GRIN2B, MFSD2A, NDE1, NTRK1, PAFAHIB1 (LIS1), RELN, and TUBB3.

33. The tissue of claim 30, wherein the cells contain more than one genomic alteration that reduces function of at least one protein respectively encoded by a gene selected from CIT, DCX, FTCD, GRIK3, GRIN2A, GRIN2B, MFSD2A, NDE1, NTRK1, PAFAH1B1 (LIS1), RELN, and TUBB3.

34. The tissue of any one of claims 30-33, wherein the cells further contain at least one genomic alteration that reduces expression of at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2;
interleukin-1 receptor family members, optionally IL1RAPL1; transcription factors, optionally SOX1, TBR1, or VAX1; potassium channels, optionally KCNK4; and PRDM12.

35. The tissue of any one of claims 30-33, wherein the cells further contain at least one genomic alteration that reduces function of the protein respectively encoded by at least one gene encoding 5HT receptors, optionally HTR6 or GPR26; dopamine receptors, optionally DRD3; GABA receptors, optionally GABRA6; glutamate receptors, optionally GRM2, GRM4, or SLC1A2; interleukin-1 receptor family members, optionally IL1RAPL1; transcription factors, optionally SOX1, TBR1, or VAX1; potassium channels, optionally KCNK4; and PRDM12.
